(19) United States
(12) Patent Application Publication
(10) Pub. No.: US 2024/0285515 A1
(43) Pub. Date: Aug. 29, 2024

(54) COSMETIC SKINCARE COMPOSITION COMPRISING PROBIOTIC MICROORGANISMS AND PREBIOTIC COMPOUNDS

(71) Applicant: The Procter & Gamble Company, Cincinnati, OH (US)

(72) Inventor: Pei Wong, New York, NY (US)

(21) Appl. No.: 18/581,735

(22) Filed: Feb. 20, 2024

Related U.S. Application Data
(63) Continuation of application No. 17/583,125, filed on Jan. 24, 2022, now abandoned.

(51) Int. Cl. A61K 8/99; A61K 8/04; A61K 8/73; A61K 8/9789; A61K 8/9794; A61Q 19/00

(52) U.S. Cl. CPC A61K 8/99; A61K 8/042; A61K 8/733; A61K 8/9789; A61K 8/9794; A61Q 19/00; A61K 2800/5922; A61K 2800/85

(57) ABSTRACT

A composition comprising at least three probiotic microorganisms, at least three prebiotic compounds, and Polymnia sonchifolia root juice. The probiotic microorganisms are preferably derived from at least Lactococcus lactis, Lactobacillus acidophilus, and Lactobacillus casei. The prebiotic compounds are preferably derived from at least Curcuma longa (turmeric) root extract, Beta vulgaris (beet) root extract, and chicory root extract. The composition may also include lactic acid as a byproduct of fermentation by Lactococcus lactis. The composition may be encapsulated allowing it to adhere to and remain on the skin more effectively and longer than without encapsulation.

### BACKGROUND

### Field of Invention

The present disclosure relates generally to compositions comprising probiotic microorganisms, and in particular to encapsulated compositions comprising probiotic microorganisms and prebiotic compounds for use in topical skincare compositions.

### Related Art

The human body comprises of trillions of microorganisms, many of which reside on the skin. Research shows that some of these microorganisms promote skin health by reinforcing the skin's natural barrier against “bad” bacteria and balancing the skin's pH levels. Many facial cleansers and antibacterial soaps are harsh on the skin, stripping it of its “good” bacteria, thereby disrupting the skin's natural microbiota. Such disruption may lead to acne, eczema, rosacea, psoriasis, and other skin problems.

Probiotics, when applied to the skin, have been shown to restore the skin's natural microbiota by acting as a protective shield against “bad” bacteria, reducing skin inflammation, and preventing premature aging, among other things. Prebiotics are compounds that promote the growth of probiotic microorganisms.

Currently in the art, topical cosmetic skincare compositions comprising probiotic microorganisms are known. What is needed is a composition with enhanced clinical benefits achieved at a lower percentage of active ingredients, where the composition adheres to the skin more readily and for a longer period.

The present disclosure relates to a composition comprising probiotic microorganisms and prebiotic compounds for use in topical skincare compositions. The composition preferably encapsulated and composed for improving the skin's smoothness, promoting cell turnover (skin exfoliation), providing antimicrobial defense by not allowing foreign pathogens to colonize the skin, locking in moisture, and strengthening the skin's barrier.

### SUMMARY OF INVENTION

It is to be understood that the phraseology and terminology employed herein are for the purpose of the description and should not be regarded as limiting.

The present disclosure is directed to a composition comprising at least three probiotic microorganisms, at least three prebiotic compounds, and Polymnia sonchifolia root juice. The probiotic microorganisms are preferably derived from at least Lactococcus lactis, Lactobacillus acidophilus, and Lactobacillus casei. The prebiotic compounds are preferably derived from at least Curcuma longa (turmeric) root extract, Beta vulgaris (beet) root extract, and chicory root extract.

The composition preferably encapsulated, said encapsulation causing the composition to produce the same or superior benefits at a lower percentage of active ingredients than a similar unencapsulated composition and causing the composition to remain on the skin longer and more effectively.

These and other features will become readily apparent upon further review of the specification, drawings, and claims.

### BRIEF DESCRIPTION OF THE DRAWINGS

Embodiments of the present invention will be described by way of example only, and not limitation, with reference to the accompanying drawings.

FIG. 1 illustrates the percentage skin penetration of free rhodamine versus rhodamine encapsulated in an alginate-lecithin nanogel, over time; and

FIG. 2 illustrates the percentage of free caffeine versus caffeine encapsulated in a biopolymeric matrix remaining on the skin after 3 hours before being absorbed.

### DETAILED DESCRIPTION

The present disclosure relates to a composition comprising at least three probiotic microorganisms and at least three prebiotic compounds as actives useful in improving the skin's appearance and preventing skin conditions caused by disruption of the skin's microbiota.

The probiotic microorganisms of the present disclosure preferably being derived from at least Lactococcus lactis, Lactobacillus acidophilus, and Lactobacillus casei, wherein the probiotic microorganisms are tyndallyzed. The composition may also comprise lactic acid resulting from the fermentation of sugars by Lactococcus lactis. Lactococcus lactis is a species of Gram-positive bacteria in the genus Lactococcus. When used in skincare products, Lactococcus lactis and the lactic acid produced thereby, have been found to stimulate cellular renewal of the skin, eliminating layers of dead skin cells and strengthening the barrier function of the skin. They also have antimicrobial properties. Lactobacillus acidophilus is a species of Gram-positive bacteria in the genus Lactobacillus. Lactobacillus acidophilus is one of the most commonly recognized species of the genus Lactobacillus and has been found to reduce acne lesions on the skin and to prevent sun-induced wrinkles. Lactobacillus casei is another species of Gram-positive bacteria in the genus Lactobacillus, and when used in skincare products has been shown to dial down inflammation to fend off infections.

Prebiotics are plant fiber compounds that induce the growth or activity of beneficial microorganisms such as probiotic microorganisms. The prebiotic compounds of the present disclosure are preferably derived from at least Curcuma longa (turmeric) root extract, Beta vulgaris (beet) root extract, and chicory root extract. Phytochemicals in Curcuma longa (turmeric) root extract, such as curcumin, are known for their anti-inflammatory and antioxidant properties. However, on its own, and in use in cosmetic applications, it exhibits poor aqueous solubility and instability. Organic solvents used for extraction of phytochemicals in Curcuma longa (turmeric) root can irritate the skin and cause contact dermatitis. As such, in the present disclosure, the phytochemicals in the Curcuma longa (turmeric) root are preferably extracted by Lactobacillus rhamnosus IDCC 3201 to create a fermentative humectant comprising water, Lactobacillus ferment, Curcuma longa (turmeric) root extract, butylene glycol and 1,2,-hexanediol. The resulting fermentative humectant having powerful anti-inflammatory, moisturizing and skin conditioning properties.

Beta vulgaris (beet) root extract is a source of phytochemicals and bioactive compounds, which are known for their roles in providing anti-aging, skin brightening, and anti-inflammatory effects to the skin. Beta vulgaris is classified as one of the ten plants with the highest antioxidant activity. Alpha-glucan oligosaccharide is present in the composition as a derivative of Beta vulgaris (beet) root extract. Generally, alpha-glucan oligosaccharide is a prebiotic derived from the enzymatic synthesis of vegetable substrates such as corn maltose and beetroot saccharose. Alpha-glucan oligosaccharide is used for the protection and bio-stimulation of natural skin defenses.

Inulin is a naturally occurring polysaccharide found in certain plants. In the present disclosure, inulin is preferably extracted from chicory root. Inulin is a prebiotic and a natural humectant drawing moisture into the skin and assisting with the prevention of acne, rosacea, and signs of premature aging.

The composition may be encapsulated for use in topical skincare compositions. Encapsulation provides the same or greater clinical benefits as the un-encapsulated composition, with the use of a lower percentage of actives. It allows the composition to adhere to and remain on the skin more effectively and longer than without encapsulation.

Encapsulation is the process of shielding a component from its environment by enclosing it in another material, such as a microcapsule or colloidal hydrogel system. Encapsulation means may be by any means known in the art. A preferred method of encapsulation is in an alginate-lecithin nanogel. Studies have shown that encapsulation of probiotic microorganisms in alginate beads containing lecithin improved the viability of the encapsulated microorganisms. Alginate is a linear unbranched polysaccharide extracted from brown seaweed and may have varying compositions when obtained from different sources. Alginate comprises of varying proportions of (1,4)-linked beta D-mannuronic acid (M) and alpha L-guluronic acid (G) residues, as shown by way of example below:

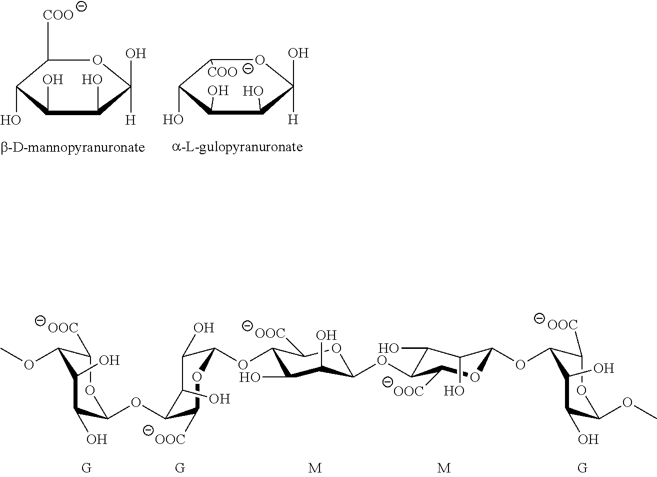

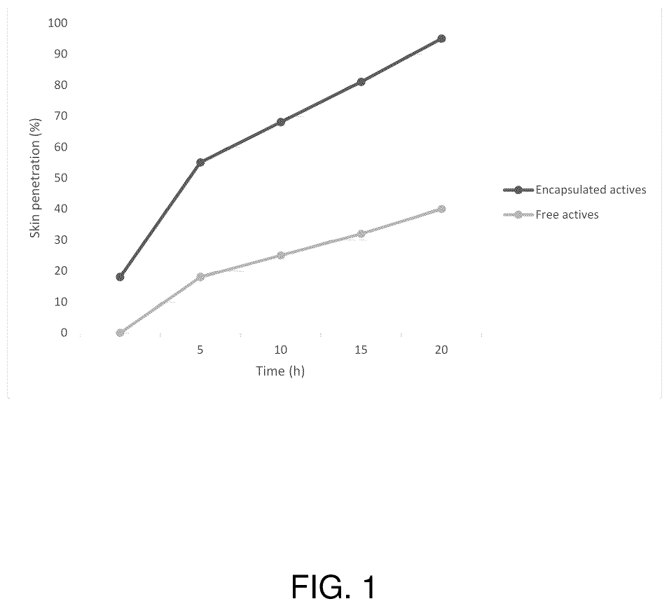

Alginate may be gelled by stacking guluronic acid blocks cross linked by cation junctions. In the present disclosure calcium is preferably used because it is considered clinically safe and readily available, however other cations may be used. The alginate forms hydrocolloids which favor the encapsulation of the hydrophilic active ingredients and the viscoelastic properties of the microcapsules provide a film effect on the skin. FIG. 1 illustrates the percentage skin penetration of free rhodamine versus encapsulated rhodamine in an alginate-lecithin nanogel over time. The alginate-lecithin nanogel allows for sustained release of the encapsulated actives, increasing the bioavailability of the encapsulated actives and decreasing the frequency of administration. The alginate-lecithin nanogel allows for further sustained release as compared to encapsulation using liposomes.

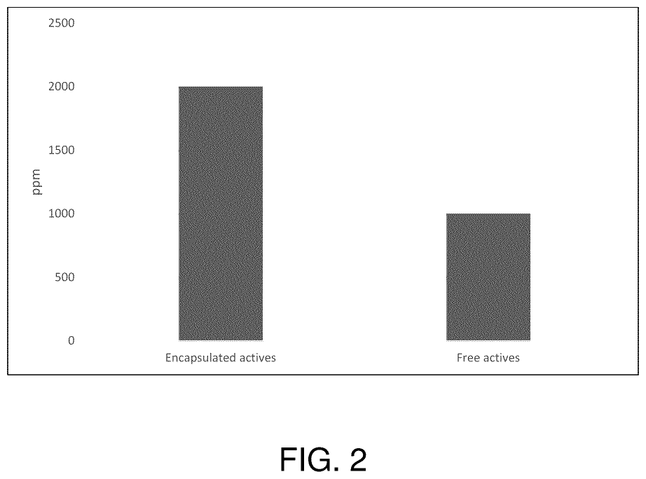

The composition may be encapsulated in a biopolymeric matrix, that when applied to the skin, allows the composition to adhere to the skin and retain the active ingredients for a longer period. FIG. 2 illustrates the percentage of free caffeine versus encapsulated caffeine in a biopolymeric matrix remaining on the skin after 3 hours before being absorbed, representing the slow release of the encapsulated active. The encapsulate comprises cellulose-based polymers and dimethyl isosorbide. Dimethyl isosorbide (DMI) enhances the penetration of the actives into the skin when the encapsulated composition is applied to the skin. The encapsulate forms a matrix on the skin, allowing the actives to remain on the skin longer. This increased skin contact time enhances the abilities of the DMI by providing it with more time in contact with the skin effectively resulting in higher absorbance of the actives into the skin.

The composition may be encapsulated in a tridimensional hydrocolloid gel matrix made of polysaccharides such as galactomannans or amylopectin. The galactomannans or amylopectin are solubilized forming a hydrocolloid pre-gel. The active ingredients are incorporated into the pre-gel through multi-capillary injection. The system is cooled using a refrigeration system, resulting in a tridimensional matrix comprising the active molecules.

Tables 1-3 show example components and formulations for preparing the composition. In particular, a first example of a composition in an unencapsulated form (“Composition 1”) may be prepared with the formulation shown in Table 1 below.

TABLE 1
Composition 1

| Component | Wt. % |
|---|---|
| Lactococcus ferment lysate | 65-80% |
| Beta vulgaris (beet) root extract | 8-10% |
| Inulin | 4-5% |
| Curcuma longa (turmeric) root extract | 3-8.1% |
| Alpha-glucan oligosaccharide | 2-7% |
| Lactic acid | 0.5-2% |
| Lactobacillus ferment | 0.5-1.5% |
| Polymnia sonchifolia root juice | 0.5-2% |
| Lactobacillus | 0.04-0.1% |

A second example of a composition encapsulated in an alginate-lecithin nanogel (“Composition 2”) may be prepared with the formulation shown in Table 2 below.

TABLE 2
Composition 2

| Component | Wt. % |
|---|---|
| Water | |
| Lactococcus ferment lysate | 20.05-30.08% |
| Glycerin | 5.6-8.4% |
| Propanediol | 4-6% |
| Beta vulgaris (beet) root extract | 2.4-3.6% |
| Inulin | 1.2-1.8% |
| Lecithin | 0.8-1.2% |
| Curcuma longa (turmeric) root extract | 0.64-0.97% |
| Lactic acid | 0.24-0.36% |
| Alpha-glucan oligosaccharide | 0.56-0.84% |
| Algin | 0.55-0.83% |
| Polymnia sonchifolia root juice | 0.15-0.23% |
| Xanthan gum | 0.12-0.18% |
| Lactobacillus ferment | 0.1-0.15% |
| Calcium chloride | 0.08-0.12% |
| Sodium chloride | 0.036-0.054% |
| Maltodextrin | 0.08-0.12% |
| Lactobacillus | 0.008-0.012% |
| Sodium benzoate | 0.07-0.11% |
| 1,2 Hexanediol | 0.82-1.22% |
| Caprylyl glycol | 0.8-1.2% |
| Butylene glycol | 0.04-0.05% |

A third example of a composition encapsulated in a biopolymeric matrix (“Composition 3”) may be prepared with the formulation shown in Table 3 below.

TABLE 3
Composition 3

| Component | Wt. % |
|---|---|
| Propanediol | 35-40% |
| Lactococcus ferment lysate | 25-30% |
| Water | 20-25% |
| Dimethyl Isosorbide | 1-5% |
| Beta vulgaris (beet) root extract | 1-5% |
| Hydroxypropylcellulose | 1-5% |
| Inulin | 1-5% |
| Hydroxypropylmethylcellulose | 0.2-0.5% |
| Pentylene Glycol | 0.2-0.5% |
| Phenoxyethanol | 0.2-0.5% |
| Lactic acid | 0.2-0.5% |
| Curcuma Longa (Turmeric) Root Extract | 0.2-0.5% |
| Alpha-glucan oligosaccharide | 0.2-0.5% |
| Caprylyl Glycol | 0.1-0.2% |
| Ethylhexylglycerin | 0.1-0.2% |
| Sodium Benzoate | 0.04-0.1% |
| Polymnia sonchifolia root juice | 0.04-0.1% |
| Sodium Chloride | 0.04-0.1% |
| Lactobacillus ferment | 0.04-0.1% |
| Maltodextrin | 0.01-0.04% |
| Butylene Glycol | 0.01-0.04% |
| 1,2-Hexanediol | 0.01-0.04% |
| Silica | 0.01-0.04% |
| Lactobacillus | 0.01-0.04% |

A fourth example of a composition encapsulated in a tridimensional hydrocolloid gel Matrix (“Composition 4”) may be prepared with the formulation shown in Table 4 below.

TABLE 2
Composition 4

| Component | Wt. % |
|---|---|
| Lactococcus ferment lysate | 40-60% |
| Glycerin | 30-50% |
| Beta vulgaris (beet) root extract | 4-7% |
| Inulin | 2-4% |
| Lactic acid | 0.4-0.7% |
| Alpha-glucan oligosaccharide | 0.3-1% |
| Caesalpinia spinosa gum | 0.3-1% |
| Polymnia sonchifolia root juice | 0.05-0.15% |
| Sodium chloride | 0.05-0.15% |
| Maltodextrin | 0.05-0.15% |
| Lactobacillus | 0.004-0.01% |
| Sodium benzoate | 0.2-0.3% |
| Potassium sorbate | 0.2-0.3% |

This disclosure is not intended to limit the invention to the particular composition disclosed, but, to the contrary, the intention is to cover all modifications, equivalents, and alternatives falling within the scope of the claims.

What is claimed is:

1. A probiotic and prebiotic composition comprising:
a. water
b. from about 20.05 to about 30.08% Lactococcus ferment lysate;
c. from about 2.4 to about 3.6% Beta vulgaris (beet) root extract;
d. from about 1.2 to about 1.8% inulin;
e. from about 0.64 to about 0.97% Curcuma longa (turmeric) root extract;
f. from about 0.24 to about 0.36% lactic acid;
g. from about 0.56 to about 0.84% alpha-glucan oligosaccharide;
h. from about 0.15 to about 0.23% Polymnia sonchifolia root juice;
i. from about 0.1 to about 0.15% Lactobacillus ferment;
j. from about 0.036 to about 0.054% sodium chloride;
k. from about 0.08 to about 0.12% maltodextrin;
l. from about 0.008 to about 0.012% Lactobacillus;
m. from about 0.07 to about 0.11% sodium benzoate;
n. from about 0.82 to about 1.22% 1,2 Hexanediol; and
o. from about 0.04 to about 0.05% Butylene glycol;
wherein the probiotic and prebiotic composition is incorporated into a composition for improving the skin's appearance.

2. The probiotic and prebiotic composition of claim 1, wherein at least a portion of the ingredients in the probiotic and prebiotic composition are encapsulated.

3. The probiotic and prebiotic composition of claim 2, wherein the probiotic and prebiotic composition is encapsulated in an alginate-based hydrogel.

4. The probiotic and prebiotic composition of claim 2, wherein the probiotic and prebiotic composition is encapsulated in a biopolymeric hydrogel.

5. The probiotic and prebiotic composition of claim 2, wherein the probiotic and prebiotic composition is encapsulated in a tridimensional polysaccharide-based hydrocolloid gel matrix.

6. The probiotic and prebiotic composition of claim 1, wherein Lactococcus is Lactococcus lactis and the lactic acid is a byproduct of fermentation by the Lactococcus lactis.

7. A probiotic and prebiotic composition comprising:
a. from about 65 to about 80% Lactococcus ferment lysate;
b. from about 8 to about 10% Beta vulgaris (beet) root extract;
c. from about 4 to about 5% inulin;
d. from about 3 to about 8.1% Curcuma longa (turmeric) root extract;
e. from about 2 to about 7% alpha-glucan oligosaccharide;
f. from about 0.5 to about 2% lactic acid;
g. from about 0.5 to about 1.5% Lactobacillus ferment;
h. from about 0.5 to about 1.5% Polymnia sonchifolia root juice; and
i. from about 0.04 to about 0.1% Lactobacillus;
wherein the probiotic and prebiotic composition is incorporated into a composition for improving the skin's appearance.

8. The probiotic and prebiotic composition of claim 7, wherein the probiotic and prebiotic composition is unencapsulated.

9. The probiotic and prebiotic composition of claim 7, wherein Lactococcus is Lactococcus lactis and the lactic acid is a byproduct of fermentation by the Lactococcus lactis.
